(19) United States
(12) Patent Application Publication
(10) Pub. No.: US 2025/0303012 A1
(43) Pub. Date: Oct. 2, 2025

(54) Multi-Configuration Spray Misting Decontamination System

(71) Applicant: TOMI ENVIRONMENTAL SOLUTIONS INC., Frederick, MD (US)

(72) Inventors: Halden Stuart SHANE, Beverly Hills, CA (US); Elissa Jessica (E.J.) SHANE, Frederick, MD (US); Johnny Sullivan CATO, Lynwood, CA (US); Reed Alexander SCOTT, Frederick, MD (US)

(21) Appl. No.: 18/864,381

(22) PCT Filed: Jun. 30, 2022
(86) PCT No.: PCT/US2022/035700

§ 371 Date: Nov. 8, 2024

Related U.S. Application Data
(60) Provisional application No. 63/339,832, filed on May 9, 2022.

(51) Int. Cl. A61L 2/24; A61L 2/22

(52) U.S. Cl. CPC A61L 2/24; A61L 2/22; A61L 2202/14; A61L 2202/25

(57) ABSTRACT

A multi-configuration spray misting decontamination control system that controls hardware designed to cleanse substantially enclosed spaces of micro-organisms. The control system utilizes multiple application sub-systems to deliver decontamination capabilities. The control system is designed for local and remote operation. The control system can be automated or manually operated. The control system is scalable to differing numbers of sensors and decontamination applicators.

This application claims priority from U.S. Provisional Patent Application No. 63/339,832, filed May 9, 2022, and from PCT/US22/35700, filed Jun. 30, 2022, which are incorporated herein by reference.

### FIELD

The present application relates generally to a multi-configuration system for decontaminating articles, enclosed spaces, and unenclosed spaces and, more particularly, to microbiological decontamination of such locations.

### BACKGROUND

Microbial species are widely distributed in our environment. Most microbial species are of little concern, because they do not damage other living organisms. However, other microbiological species may infect man or animals and cause them harm. The removal of micro-organisms and decontamination of articles and spaces therefrom has long been of interest. Drugs and medical devices are sterilized and packaged in sterile containers.

Medical environments such as operating rooms, wards, and examination rooms are decontaminated by various cleaning procedures so that micro-organisms of concern cannot spread from one patient to another.

Many available technologies for controlling micro-organisms are of value in the context of biological warfare and bioterrorism. Furthermore, existing decontamination technologies are limited in their effectiveness in tightly enclosed environments.

Addressing the timing, duration and control of decontamination processes is technically challenging. Control systems often rely on largely human personnel on-site to monitor and conduct the decontamination process. Furthermore, systems for control of decontamination processes are neither scalable nor modular. Consequently, decontamination processes typically require a re-design of system controls, depending on the nature of the decontamination process. In view of the foregoing, there is a pressing need for a scalable and modular system that can provide time-sensitive and environmentally-sensitive control of decontamination processes even in absence of an on-site human presence.

### SUMMARY

An aspect of the application is a multi-configuration system for decontamination comprising: a general-purpose computer; a sensor package; one or more control boards; one or more applicators; and an operator device; wherein the general-purpose computer is networked by one or more control board(s) to the sensor package, wherein the sensor package can detect the presence of micro-organisms; and the sensor package is networked to the one or more applicators, wherein the one or more applicators are configured to apply a decontamination process to remove micro-organisms; and the operator device is networked to the general-purpose computer via an application programming interface (API) gateway, wherein the operator device displays a network interface to an operator; and the API gateway provides access to a system controller.

In certain embodiments, the system controller comprises a set of sub-systems, and wherein the set of sub-systems are linked by a bi-directional interface to the system controller; and the set of sub-systems comprises: an alerts sub-system, wherein when the sensor package detects the presence of a micro-organism and then the alerts sub-system alerts the operator by a display on the network interface; and a device driver sub-system, wherein the device driver sub-system networks by a uni-directional interface to the sensor package and the applicators.

In certain embodiments, the system is manually controlled by one or more individuals via the operator device. In certain embodiments, the system is under event driven control by the system controller, wherein the system controller receives an alert to the presence of micro-organisms. In certain embodiments, the system is under remote control by one or more individuals via the operator device. In certain embodiments, the applicators begin a decontamination cycle when instructions are received from the system controller via the device driver sub-system. In certain embodiments, the set of sub-systems further comprises an events sub-system. In certain embodiments, the set of sub-systems further comprises a reporting sub-system. In certain embodiments, the set of sub-systems further comprises a configuration sub-system. In certain embodiments, the set of sub-systems further comprises a software development kit. In certain embodiments, the sensor package comprises one or more control boards networked to sensors and networked to the general-purpose computer. In certain embodiments, the sensors are one or more selected from the group comprising photosensors, voltaic sensors, weight sensors, moisture sensors, and pressure sensors. In certain embodiments, the general-purpose computer comprises a single computer control board.

An aspect of the application is a non-transitory, tangible computer-readable medium comprising instructions to decontaminate a substantially enclosed space, comprising a routine of set instructions for causing a system as described herein to perform the steps of: detecting a micro-organism's presence in a substantially enclosed space, wherein the presence of the micro-organism is sensed by one or more sensors that are present within the substantially enclosed space; alerting a system controller to the presence of the microorganism in the substantially enclosed space, wherein the system controller is networked to the one or more sensors; informing an operator device of the presence of the micro-organism in the substantially enclosed space, wherein the operator device is networked to the system controller; initiating a decontamination process to remove the presence of the micro-organism in the substantially enclosed space, wherein the decontamination process is applied by one or more applicators networked to the system controller, and further wherein the one or more applicators are present in the substantially enclosed space; and further wherein the system controller initiates the decontamination process by the one or more applicators after the step of ordering the initiation of a decontamination process by an event sub-system, wherein the event sub-system is a non-transitory tangible computer-readable medium comprising a set of instructions for causing the one or more applicators to initiate the decontamination process after the one or more sensors have detected the presence of a specific micro-organism.

In certain embodiments, the specific micro-organism is a pathogen. In certain embodiments, the pathogen is a targeted bioterror agent. In certain embodiments, the targeted bioterror agent is selected from the group consisting of anthrax (Bacillus antracis), plague (Yersinia pestis), and tularemia (Franciscella tularensis). In certain embodiments, the operator device is networked wirelessly to the system controller.

An aspect of the application is a method of controlling decontamination of a substantially enclosed space comprising: detecting a micro-organism's presence in a substantially enclosed space, wherein the presence of the micro-organism is sensed by one or more sensors that are present within the substantially enclosed space; alerting a system controller to the presence of the micro-organism in the substantially enclosed space, wherein the system controller is networked to the one or more sensors; informing an operator device of the presence of the micro-organism in the substantially enclosed space, wherein the operator device is networked to the system controller; initiating a decontamination process to remove the presence of the micro-organism in the substantially enclosed space, wherein the decontamination process is applied by one or more applicators networked to the system controller, and further wherein the one or more applicators are present in the substantially enclosed space; and further wherein the system controller initiates the decontamination process by the one or more applicators after the steps of either: (1) ordering the initiation of a decontamination process from the operate device; or (2) ordering the initiation of a decontamination process by an event sub-system, wherein the event sub-system is a non-transitory tangible computer-readable medium as described herein.

In certain embodiments, the man-made structure is an office building.

### BRIEF DESCRIPTION OF THE DRAWINGS

FIG. 1 shows the architecture of the system components and their relationships.

FIG. 2 shows the layered architecture of the system.

FIG. 3 shows an exemplary embodiment of the end user interface and administrator interface.

FIG. 4 shows the high level computer system architecture.

FIG. 5 shows the Application Runtime organizational block diagram.

FIG. 6 shows the Device Driver Sub-system event state change design.

FIG. 7 shows a systems flow chart.

While the present disclosure will now be described in detail, and it is done so in connection with the illustrative embodiments, it is not limited by the particular embodiments illustrated in the figures and the appended claims.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### Definitions

As used in this specification and the appended claims, the singular forms “a,” “an” and “the” include plural referents unless the content clearly dictates otherwise.

As used herein, the term “decontaminating” or “decontamination” means acting to neutralize or remove pathogens from an area or article.

As used herein, the terms “micro-organism” or “pathogen” include, but are not limited to, a bacterium, fungus, yeast, protozoan, virus, or other microorganisms. The term “pathogen” also encompasses targeted bioterror agents.

As used herein, the term “bacteria” shall mean members of a large group of unicellular microorganisms that have cell walls but lack organelles and an organized nucleus. Synonyms for bacteria may include the terms “microorganisms”, “microbes”, “germs”, “bacilli”, and “prokaryotes.” Exemplary bacteria include, but are not limited to Mycobacterium species, including M. tuberculosis; Staphylococcus species, including S. epidermidis, S. aureus, and methicillin-resistant S. aureus; Streptococcus species, including S. pneumoniae, S. pyogenes, S. mutans, S. agalactiae, S. equi, S. canis, S. bovis, S. equinus, S. anginosus, S. sanguis, S. salivarius, S. mitis; other pathogenic Streptococcal species, including Enterococcus species, such as E. faecalis and E. faecium; Haemophilus influenzae, Pseudomonas species, including P. aeruginosa, P. pseudomallei, and P. mallei; Salmonella species, including S. enterocolitis, S. typhimurium, S. enteritidis, S. bongori, and S. choleraesuis; Shigella species, including S. flexneri, S. sonnei, S. dysenteriae, and S. boydii; Brucella species, including B. melitensis, B. suis, B. abortus, and B. pertussis; Neisseria species, including N. meningitidis and N. gonorrhoeae; Escherichia coli, including enterotoxigenic E. coli (ETEC); Vibrio cholerae, Helicobacter pylori, Geobacillus stearothermophilus, Chlamydia trachomatis, Clostridium difficile, Cryptococcus neoformans, Moraxella species, including M. catarrhalis, Campylobacter species, including C. jejuni; Corynebacterium species, including C. diphtheriae, C. ulcerans, C. pseudotuberculosis, C. pseudodiphtheriticum, C. urealyticum, C. hemolyticum, C. equi; Listeria monocytogenes, Nocardia asteroides, Bacteroides species, Actinomycetes species, Treponema pallidum, Leptospirosa species, Klebsiella pneumoniae; Proteus sp., including Proteus vulgaris; Serratia species, Acinetobacter, Yersinia species, including Y. pestis and Y. pseudotuberculosis; Francisella tularensis, Enterobacter species, Bacteroides species, Legionella species, Borrelia burgdorferi, and the like. As used herein, the term “targeted bioterror agents” includes, but is not limited to, anthrax (Bacillus antracis), plague (Yersinia pestis), and tularemia (Franciscella tularensis).

As used herein, the term “virus” can include, but is not limited to, influenza viruses, herpesviruses, polioviruses, noroviruses, and retroviruses. Examples of viruses include, but are not limited to, human immunodeficiency virus type 1 and type 2 (HIV-1 and HIV-2), human T-cell lymphotropic virus type I and type II (HTLV-I and HTLV-II), hepatitis A virus, hepatitis B virus (HBV), hepatitis C virus (HCV), hepatitis delta virus (HDV), hepatitis E virus (HEV), hepatitis G virus (HGV), parvovirus B19 virus, hepatitis A virus, hepatitis G virus, hepatitis E virus, transfusion transmitted virus (TTV), Epstein-Barr virus, human cytomegalovirus type 1 (HCMV-1), human herpesvirus type 6 (HHV-6), human herpesvirus type 7 (HHV-7), human herpesvirus type 8 (HHV-8), influenza type A viruses, including subtypes H1N1 and H5N1, human metapneumovirus, severe acute respiratory syndrome (SARS) coronavirus, hantavirus, and RNA viruses from Arenaviridae (e.g., Lassa fever virus (LFV)), Pneumoviridae (e.g., human metapneumovirus), Filoviridae (e.g., Ebola virus (EBOV), Marburg virus (MBGV) and Zika virus); Bunyaviridae (e.g., Rift Valley fever virus (RVFV), Crimean-Congo hemorrhagic fever virus (CCHFV), and hantavirus); Flaviviridae (West Nile virus (WNV), SARS-CoV-2 and variants, Dengue fever virus (DENV), yellow fever virus (YFV), GB virus C (GBV-C; formerly known as hepatitis G virus (HGV)); Rotaviridae (e.g., rotavirus), and combinations thereof. In one embodiment, the subject is infected with HIV-1 or HIV-2. As used herein, the term “fungi” shall mean any member of the group of saprophytic and parasitic spore-producing eukaryotic typically filamentous organisms formerly classified as plants that lack chlorophyll and include molds, rusts, mildews, smuts, mushrooms, and yeasts. Exemplary fungi include, but are not limited to, Aspergillus species, Dermatophytes, Blastomyces derinatitidis, Candida species, including C. albicans and C. krusei; Malassezia furfur, Exophiala werneckii, Piedraia hortai, Trichosporon beigelii, Pseudallescheria boydii, Madurella grisea, Histoplasma capsulatum, Sporothrix schenckii, Histoplasma capsulatum, Tinea species, including T. versicolor, T. pedis T. unguium, T. cruris, T. capitus, T. corporis, T. barbae; Trichophyton species, including T. rubrum, T. interdigitale, T. tonsurans, T. violaceum, T. yaoundei, T. schoenleinii, T. megninii, T. soudanense, T. equinum, T. erinacei, and T. verrucosum; Mycoplasma genitalia; Microsporum species, including M. audouini, M. ferrugineum, M. canis, M. nanum, M. distortum, M. gypseum, M. fulvum, and the like.

As used herein, the term “protozoan” shall mean any member of a diverse group of eukaryotes that are primarily unicellular, existing singly or aggregating into colonies, are usually nonphotosynthetic, and are often classified further into phyla according to their capacity for and means of motility, as by pseudopods, flagella, or cilia. Exemplary protozoans include, but are not limited to Plasmodium species, including P. falciparum, P. vivax, P. ovale, and P. malariae; Leishmania species, including L. major, L. tropica, L. donovani, L. infantum, L. chagasi, L. mexicana, L. panamensis, L. braziliensis and L. guyanensi; Cryptosporidium, Isospora belli, Toxoplasma gondii, Trichomonas vaginalis, and Cyclospora species.

As used herein, the term “article” means any solid item or object that may be susceptible to contamination with pathogens. As used herein, the term “substantially enclosed space” means a room, a tent, a building, or any man-made structure that is substantially enclosed and may be susceptible to contamination with pathogens. The term “substantially enclosed space” is not limited to man-made structures (e.g., caves or natural tunnels are also substantially enclosed spaces), even though embodiments illustrated herein may be preferably directed to decontamination of such structures

As used herein, the term “sensor” can refer to any type of sensor suitable for detecting contamination on an apparatus, a surface, or in a substantially enclosed space. Examples of sensors include, but are not limited to, photosensors, voltaic sensors, weight sensors, moisture sensors, pressure sensors, or any type of biosensor.

As used herein, an “enclosed space” refers to any chamber, container or space that can be decontaminated with the system of the present disclosure. Examples of enclosed spaces include, but are not limited to, any chamber used in everyday to conduct highly controlled research projects/spaces, sanitation chambers (such as gynoprobe cabinets), biosafety cabinets, glovebox, research hoods and clinical spaces.

As used herein, a “computer” may be either a general-purpose computer or a specialized device built to solely carry out one or more specific purposes.

As used herein, an “applicator” may be any form of device that can carry out a decontamination process. In particular embodiments, applicators apply decontamination processes by spray misting a substantially enclosed space.

An aspect of the application relates to a multi-configuration system for decontamination, comprising: one or more sensors, one or more applicators and a system controller, wherein when the one or more sensors detect the presence of a micro-organism, the system controller orders the one or more applicators to initiate a decontamination process.

One aspect of the application relates to a multi-configuration system for decontamination, comprising: a general-purpose computer; a sensor package; one or more control boards; one or more applicators; and an operator device; wherein the general-purpose computer is networked by one or more control board(s) to the sensor package, wherein the sensor package can detect the presence of micro-organisms; and the sensor package is networked to the one or more applicators, wherein the applicators can apply a decontamination process to remove micro-organisms; and the operator device is networked to the general-purpose computer via an application programming interface (API) gateway, wherein the operator device displays a network interface to an operator; and the API gateway provides access to a system controller. In particular embodiments, the system controller comprises a set of sub-systems, wherein the set of sub-systems are linked by a bi-directional interface to the system controller; and the set of sub-systems comprises: an alerts sub-system, wherein when the sensor package detects the presence of a micro-organism and then the alerts sub-system alerts the operator by a display on the network interface; and a device driver sub-system, wherein the device driver sub-system networks by a uni-directional interface to the sensor package and the applicators.

In certain embodiments, the system is manually controlled by one or more individuals via the operator device. In other embodiments, the system is under event driven control by a system controller, wherein the system controller receives an alert to the presence of pathogens in the environment. In specific embodiments, the system is under remote control by one or more individuals via the operator device. In further embodiments, the applicators begin a decontamination cycle when instructions are received from the system controller via the device driver sub-system. In particular embodiments, the set of subsystems further comprises an events sub-system. In particular embodiments, the set of subsystems further comprises a reporting sub-system. In particular embodiments, the set of subsystems further comprises a configuration sub-system. In particular embodiments, the set of sub-systems further comprises a software development kit. In particular embodiments, the sensor package comprises one or more control boards linked to sensors and linked to the general-purpose computer. In particular embodiments, the sensors are one or more selected from the group comprising photosensors, voltaic sensors, weight sensors, moisture sensors, and pressure sensors. In particular embodiments, the general-purpose computer comprises a single computer control board.

Another aspect of the application relates to a non-transitory, tangible computer-readable medium comprising instructions to decontaminate a substantially enclosed space, comprising a routine of set instructions for causing a multi-configuration system for decontamination to perform the steps of detecting a micro-organism's presence in a substantially enclosed space, wherein the presence of the micro-organism is sensed by one or more sensors that are present within the substantially enclosed space; alerting a system controller to the presence of the micro-organism in the substantially enclosed space, wherein the system controller is networked to the one or more sensors; informing an operator device of the presence of the micro-organism in the substantially enclosed space, wherein the operator device is networked to the system controller; initiating a decontamination process to remove the presence of the micro-organism in the substantially enclosed space, wherein the decontamination process is applied by one or more applicators networked to the system controller, and further wherein the one or more applicators are present in the substantially enclosed space; and further wherein the system controller initiates the decontamination process by the one or more applicators after the step of ordering the initiation of a decontamination process by an event sub-system, wherein the event sub-system is a non-transitory tangible computer-readable medium comprising a set of instructions for causing the one or more applicators to initiate the decontamination process after the one or more sensors have detected the presence of a specific micro-organism.

A further aspect of the application relates to a method of controlling decontamination of a substantially enclosed space, comprising: detecting a micro-organism's presence in a substantially enclosed space, wherein the presence of the micro-organism is sensed by one or more sensors that are present within the substantially enclosed space; alerting a system controller to the presence of the micro-organism in the substantially enclosed space, wherein the system controller is networked to the one or more sensors; informing an operator device of the presence of the micro-organism in the substantially enclosed space, wherein the operator device is networked to the system controller; initiating a decontamination process to remove the presence of the micro-organism in the substantially enclosed space, wherein the decontamination process is applied by one or more applicators networked to the system controller, and further wherein the one or more applicators are present in the substantially enclosed space; and further wherein the system controller initiates the decontamination process by the one or more applicators after the steps of either: (1) ordering the initiation of a decontamination process from the operate device; or (2) ordering the initiation of a decontamination process by an event sub-system, wherein the event subsystem is a non-transitory tangible computer-readable medium comprising instructions to decontaminate a substantially enclosed space

### System Architecture

An aspect of the application is a multi-configuration spray misting decontamination control system. The control system is a multi-component application that controls hardware. The control system utilizes multiple application sub-systems to deliver capabilities. The control system is designed for local and remote operation and uses a small application runtime that is controlled by a front-end web experience.

The control system is designed to be run standalone or connected to a network. Control system connectivity is facilitated using IP connectivity via wireless (WiFi/Bluetooth) or wired connectivity. IP addressing is configurable at the operating system level via browser Interface. The control system operation is governed by an application runtime, system controller, and web services layer. Components of the system may include: a mobile and web application framework; input/output (I/O) Internet of Things (IoT) boards; user authentication and identity management; high speed, web server; high speed, Java script web runtime; node package manager; and general purpose operating system (e.g. Linux, etc).

In an exemplary embodiment, the computer system includes a memory, a processor, and, optionally, a secondary storage device. In some embodiments, the computer system includes a plurality of processors and is configured as a plurality of, e.g., bladed servers, or other known server configurations. In particular embodiments, the computer system also includes an input device, a display device, and an output device. In some embodiments, the memory includes RAM or similar types of memory. In particular embodiments, the memory stores one or more applications for execution by the processor. In some embodiments, the secondary storage device includes a hard disk drive, floppy disk drive, CD-ROM or DVD drive, or other types of non-volatile data storage. In particular embodiments, the processor executes the application(s) that are stored in the memory or the secondary storage, or received from the internet or other network. In some embodiments, processing by the processor may be implemented in software, such as software modules, for execution by computers or other machines. These applications preferably include instructions executable to perform the functions and methods described herein. The applications preferably provide GUIs through which users may view and interact with the application(s). In other embodiments, the system comprises remote access to control and/or view the system.

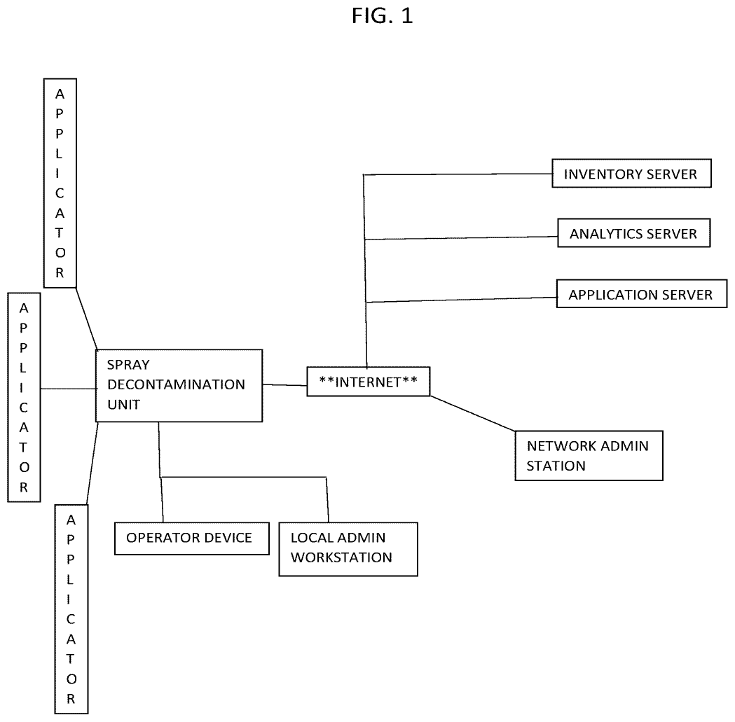

The control system is a computer-implemented control system that can deliver decontamination processes to rooms and other areas using various applicators, FIG. 1 shows an embodiment of the architecture of the system components and their relationships. In this embodiment, the control system utilizes a general-purpose computer. The control system is operated by the general-purpose computer. The general-purpose computer does not require specialized hardware and provides the greatest flexibility of programming languages, development environments, and software and accessory support. Furthermore, a general-purpose computer provides reliable I/O, an easily installable operating environment, and lowest cost.

The general-purpose computer interfaces to sensors, event signals, and decontaminating solution applicators via multiple control boards. One of ordinary skill will understand that the number of applicators is not limiting on the system. In one embodiment, the system uses up to twenty applicators. However, other embodiments may use up to five applicators, up to ten applicators, up to fifteen applicators, up to thirty applicators, up to forty applicators, up to fifty applicators, up to one hundred applicators, etc. The number of applicators used is ultimately dictated by the size of the required decontamination project, e.g., a decontamination system that is fitted to a large multi-floor office building may have multiple applicators on every floor. The control system herein supports all configurations and supports individual, manual operation, event driven control, and remote controlled operation. In a preferred embodiment, the applicators use binary ionization technology for decontamination, which uses high voltage current to ionize hydrogen peroxide to decontaminate an area. Applicators are available in portable units for decontaminating surfaces, which use a handheld applicator; environmental units, which are case-contained, transportable devices; and fixed units, which are centrally installed and controlled in substantially enclosed spaces (e.g., inside office buildings or laboratories).

The general-purpose computer is networked to an operator device and a local administrator workstation. Operation of the control system can be controlled using any operator device with a suitable web browser. In certain embodiments, the operator device may be physically integrated with the general purpose computer (2). In other embodiments, the operator device may be remotely linked to the general purpose computer by a wireless network connection. In a particular embodiment, the control system is designed for remote operation via its web application. The application software is designed to be extensible by exposing a software development kit (SDK) that can be used for building enhanced applications without requiring a redeployment of the software. The SDK provides the ability to access the control system using Representational State Transfer (RESTful) web services to support remote and custom applications.

In some embodiments, the local administrator workstation is directly networked to the general-purpose computer via suitable cables. In other embodiments, the local administrator workstation may be remotely linked to the general-purpose computer. In particular embodiments, the local administrator workstation may not be included, or, in an alternative embodiment, an operator device may be used as the local administrator workstation. The general purpose computer is also connected by an internet connection to a third party administrator workstation, which may be used by a third party that is responsible for maintaining the control system and overseeing any decontamination operations. In certain embodiments, the third party administrator workstation is not included, or, in an alternative embodiment, the third party administrator workstation is an operator device. The general purpose computer is also connected by an internet connection to an inventory server, an analytics server and an application server.

In one embodiment, the control system runs on a single board, general-purpose computer with embedded RAM, I/O, and chipset. In a particular embodiment, the general-purpose computer consists of a single board that can be easily mounted in a container.

The control system interfaces with a number of sensors, switches, and event driven mechanisms. In one embodiment, the computer hardware design incorporates the control boards for the sensor package. The applicators are connected by a sensor package to the general purpose computer. The sensor package is controlled using off-the-shelf, control boards with multiple general-purpose I/O ports (GPIO) that can be used to interface with multiple devices. The boards are designed to support the Internet of Things (IoT) and are easily flashed with custom software or can be controlled externally. The control boards interface with the control system via USB and are controlled using a specialized serial driver loaded by the operating system. A control board is programmable so that it can be easily configured to support data from flow meters, temperature, humidity, light level and most any imaginable sensor that provides voltage, pulse, frequency, digital or current outputs. It can also provide digital and analog outputs, as well as PWM (pulse width modulation) output for controlling many types of actuators (e.g., the decontaminating devices which apply spray mist to a space or article for decontamination) and devices. To support connectivity to the general-purpose computer, a small application runs on the control board and manages the low-level interfaces and any required data normalization. The application makes available a series of commands that would be executed over the USB connection from the main system controller. These commands would provide for maintenance tests of the sensors and actuators, reading of sensor data and control of the output actuators. As needed, an interface Printed Circuit Board (PCB) would be added that simplifies the connection between the control board and the control system flow meter and applicator hardware, as well as any additional temperature or humidity sensors.

In an exemplary embodiment, the control system runs on an Ubuntu Linux operating system (e.g., open source software). Ubuntu is used on the desktop, server hardware, and embedded systems. Ubuntu is not a real time operating system but is capable of measuring short time slices and is highly responsive. Ubuntu is hardened by activating its installed firewall, ufw, and closing all unnecessary ports.

In certain embodiments, Secure Shell (SSH) access is the primary means of direct access to the computer and is primarily used for installation and deployment, and upgrading various software components such as the operating system. Certain configuration settings may be set at the operating system level. These settings typically include the date and time and administrative authentication (LDAP). In certain embodiments, automatic time synchronization and the sendmail configuration are the only two settings recommended to be set at the operating system level.

Data normalization and filtering can also be performed on this module as well, providing clean sensor data to the system's main controller. The platform can interface to a computer system or board via a standard USB interface. This interface appears as a virtual serial port (COM port) to the main controller. The virtual serial port makes it very simple to issue commands between the computer and underlying hardware at the software level.

Commands and responses are transmitted between the computer driver and hardware as ASCII text. Nearly all modem languages and runtimes support the transmission of ASCII text over a COM port.

### End-User Implemented Processes

The control system software follows a layered architectural approach. Applications, interfaces, and their services are built on top of supporting components and frameworks providing abstractions from lower level functionality. This approach provides the greatest degree of flexibility for deploying updated software and installing new components. This is possible because the layers of abstraction allow the implementation of supporting services and sub-systems to change without requiring updates between the layers unless absolutely necessary.

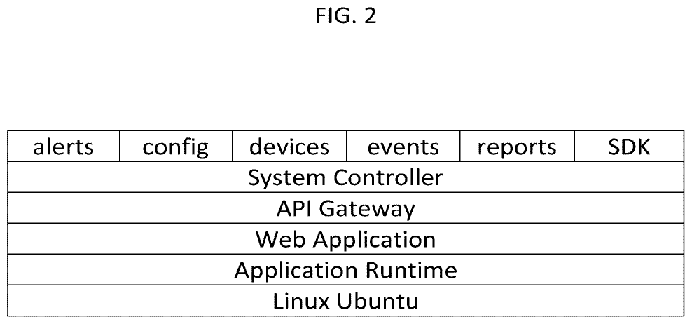

FIG. 2 shows an embodiment of the layered architecture. The architecture consists of six layers. The first layer, with which the user will interface, contains six subsystems, which comprise alerts, configuration, devices, events, reports and the software developer kit. The second layer is the system controller, which is the general-purpose computer. The third layer is the application programming interface (API) gateway by which the general-purpose computer is networked to the other components of the system. The fourth layer is the web application by which the networked communications within the system occur. The fifth layer is the application runtime which executes the instructions communicated within the system. The sixth layer is the coding language.

The System Controller provides a layer of abstraction between the control system features and functionality and applications. The System Controller controls the system. The System Controller is designed as a mid-level component that is memory-loaded and accepts commands from internal and external systems. The System Controller does maintain state as well as governing system configuration. It operates in its own process space and executes as a fault tolerant, automatic service. It is configured to be auto-enabled, and will auto start on computer boot up. The System Controller governs the control system startup and shutdown operations. The System Controller interfaces with multiple subsystems to govern the control system operation. Interfacing with sub-systems is restrained in that the System Controller doesn't make direct access to the underlying low-level hardware, reporting, configuration or other sub-system implementation. These layers of abstraction provide a flexible architecture that gives the user the ability to switch out hardware or change the implementation of any sub-system without requiring significant (or any) change to the System Controller or operation of the control system. As stated, the System Controller itself provides abstraction to the API Gateway and Web Server components so that it can upgraded or changed without requiring significant rework or change to other components.

The Web Server component serves a web application that is used as the command and control system for the control system. Operators are provided with a username and password and login to the control system to make configuration settings, start applicator runs, and download reports and logs.

In general, all application specific configuration parameters and access to features will be made available via the web application. This includes the ability to specify event dispatches, time and timer based operations, report generation configurations, and room/applicator profiles, and user access. Configuration parameters and layout will be determined in the web application user interface design and style guidelines documentation.

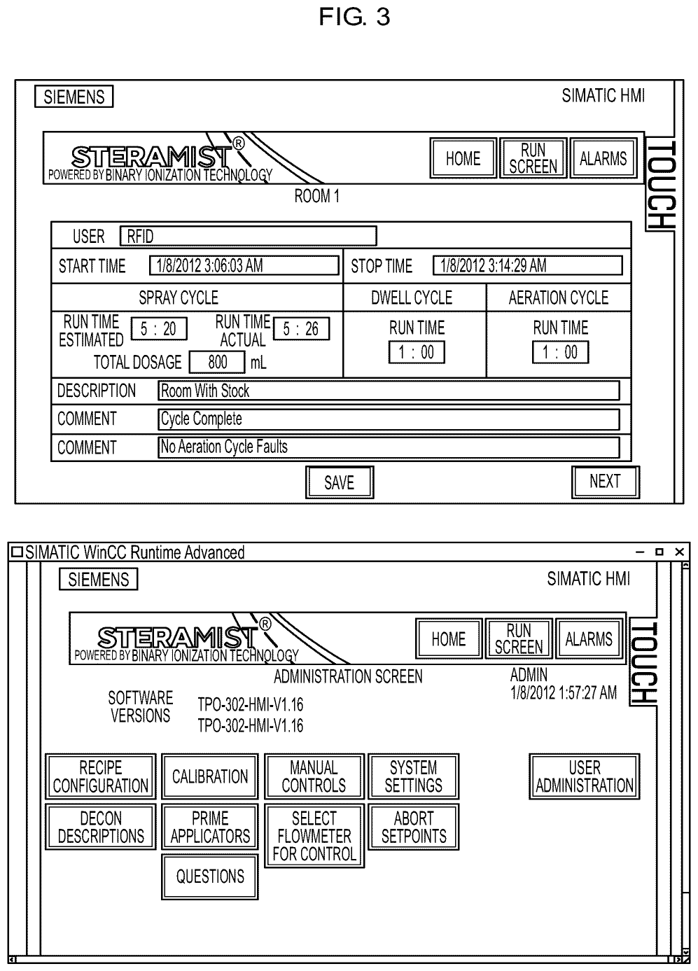

FIG. 3 shows an exemplary embodiment of the end user interface and administrator interface. The interface would accommodate both polling as well as interrupt or exception based reporting to the main system controller. The Log In screen will be presented initially to request credentials of a user to reach the system home page. Options to start a decontamination cycle will be given. Control and settings options allow for recipe configurations, calibration, priming, controls and settings parameters to be adjusted. Recipe configuration allows for recipe selection, room dimensions adjustment, spray cycle settings, RABS and LAF settings, time countdown and H2O2 sensor reading setpoints. Room descriptions allow for manual descriptions of each room to be decontaminated. Calibration allows dose verification and fluid rate calibration per applicator. Priming of applicators allows selection of applicators to be primed at high or normal speed. Safety questions can be enabled or disabled and text modified. Manual controls of pumps and valves for each applicator are provided. Flow meter controls allow flow meter selection at gear pump or applicator for each. Systems settings enable or disable features, and allow date/time adjustments. Parameters to abort setpoints may be set to allow tolerance for fault generation. User administration allows for new users, user level settings and passwords.

Once a start cycle button is pressed, a recipe must be selected to run. Descriptions and permissive questions will need selection if a feature is enabled in settings. An “initiate cycle” button will appear once all once permissive are met. The run screen will show all cycle information once initiated. Report screens will show report information on the course of each cycle after that cycle ends (whether successfully or unsuccessfully).

The system will monitor supplies of decontaminating solution via a decontaminating solution indicator. A “low tank level” message will appear in yellow on Log in and home screens if the decontamination solution storage tank needs more fluid. If shortage of decontamination solution is not addressed, “reservoir tank not filling” message will display in red. To clear message, a user must select “pump control” on control and settings menu and activate the fluid pump until “high level status” indicator turns green. (Decontamination solution volume shortage should be addressed to prevent reoccurrence).

In certain embodiments, hardware interface commands may include ordering a self-test of all connected sensors with a pass or fail response; a calibration process for all sensors; a read back display of current sensor values for each sensor; set sample rate for each sensor; set scaling parameters for each sensor; self-test for all connected actuators; control calibration process for actuators; set output change rates for actuators; energize actuators; disengage actuators; set all parameters to a suggested default; and clear all settings.

### Computer-Implemented Processes

An aspect of the application is a control system that is a software application that controls hardware. Therefore, the software must be expedient, highly responsive, and reactive to external signals and events. The control system software is architected to take up a very small footprint and uses a simplified design consisting of an application runtime and multiple systems that support control system features.

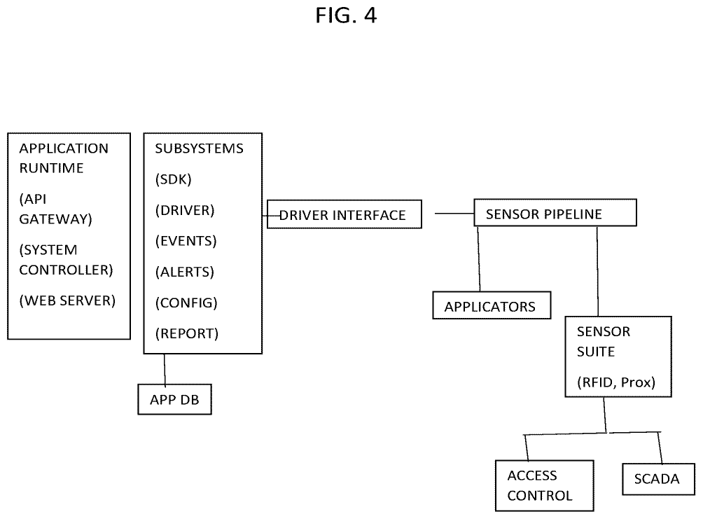

FIG. 4 shows one embodiment of the high level computer system architecture. The Application Runtime consists of three primary services. An API Gateway used for standardized access to the control system control and configuration, a low-level System Controller, and a Web Server that provides user remote access. The API Gateway and Web Server services both utilize the System Controller to make configuration modifications, calibrate sensors, and control the system. The Application Runtime uses many services to perform its work. Each service is designed to be fault tolerant in that it will automatically restart after failure and is configured to auto start at boot time.

The API Gateway serves as the application endpoint for all internal and external systems that configure and control the decontamination control system. The API Gateway is a running service installed on the computer and accessible via IP address and port number. Internal applications and systems will reference the API Gateway in the same manner as external services, except that all internal communications will be connected via local host. The API Gateway can be started and stopped independently providing the ability for its software to be updated. The API Gateway is stateless meaning that it accepts incoming requests and processes them without saving any local data. It exposes several RESTful services that are mapped to configuration and control functions as outlined by the feature set. An API gateway provides a cost effective platform that provides the ability of extending the system without significant re-engineering. The primary means of communicating with the general-purpose computer is via its API Gateway. The API Gateway will expose many endpoints that are used for configuration as well as monitoring and data capture. Bi-direction communications will be facilitated via web hook for any applications that require asynchronous delivery of data from the control system.

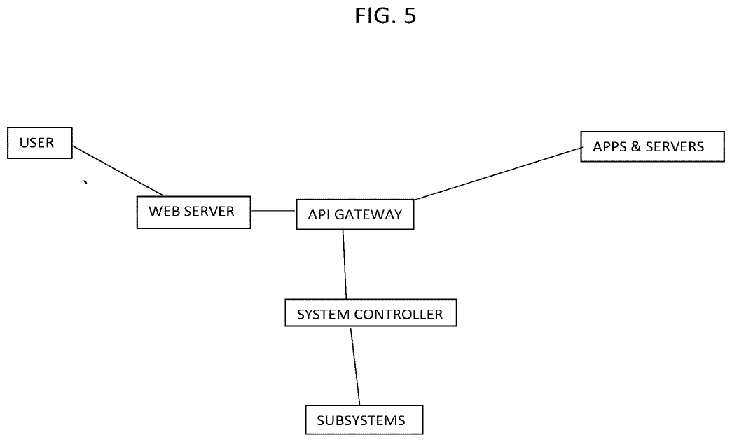

FIG. 5 shows one embodiment of the Application Runtime organizational block diagram. The user is linked by the web server through the API gateway to the system controller and the sub-systems. The API gateway also provides a networked link to the web applications and other servers, etc.

Further, also in the embodiment shown in FIG. 4, are the sub-systems, which are alerts, configuration, devices, events, reports and the software developer kit are also part of an application database on the general computer. In addition, the sub-systems connect via a driver interface to a sensor pipeline, which is linked to the applicators and a sensor suite.

The sub-systems herein are a collection of the lowest level execution components that have direct access to hardware features and functionality. Each sub-system is designed for a major grouping of features in the environment.

In one embodiments, a general sub-system design comprises each sub-system having two interfaces. One interface provides a high-level of abstraction that is best suited for applications and higher order programs; this interface links the system controller with the sub-system. This interface is bi-directional in nature, meaning that the sub-system accepts and executes functions. The second interface is a low-level, one directional interface that is specifically used to communicate with underlying hardware or deliver low-level functionality.

Sub-systems are purposefully designed to be autonomous components. This provides the ability to make software changes to a specific sub-system and have the single sub-system patched to the solution without requiring monolithic changes to be made to the solution.

### Alerts Sub-System

The Alerts Sub-system is used for processing all outbound messages from the control system.

Alerts are implemented as messages with a recipient address, a sending address, and a body consisting of UTF-8 text. Alerts may comprise simple text, email messages to designated recipients or lists, and notifications internal to application services and other sub-systems.

Email notifications require a specified mailbox and SMTP configuration. Operators will set SMTP parameters including the mailbox, and username, password credentials using the configuration application accessible by web browser. Emails can be delivered using the built-in sendmail application or programmatically using Javax or NodeJS libraries.

Sub-systems and internal services utilize the Alerts Sub-system by registering itself as a publisher and stating the types of messages and delivery mechanisms. Internal services can register for Alerts by registering themselves as an observer to a sub-system's specific messages. The common, Producer/Consumer design pattern is used to automatically dispatch messages to interested parties.

External systems that require alerts also utilize the Alerts Sub-system. The same mechanisms are used to register themselves as a message receiver, except that external services use the API Gateway's RESTful service interface. External services must supply a webhook that can be called by Alerts Sub-system. This means that an external service is technically a consumer as well as a producer.

In certain embodiments, external alert systems including SMS are not supported, but can be implemented using RESTful webservices or other means.

The Producer/Consumer model used in the Alerts Sub-system is designed in a generic fashion so that it can be reused in other control system components that require a registration and publication framework.

### Configuration Subsystem

The Configuration Sub-system is the command processor for storing and modifying configuration values in the system.

The Configuration Sub-system uses an embedded database to store its values. A runtime database that runs in its own thread and process space will be avoided. A separate runtime database adds increased installation and deployment complexity. Most file-based embedded databases can handle gigabytes of data and millions of records, which are more than suitable for the control system. Furthermore, an embedded database provides simpler backup and restoration methods.

In most cases configuration parameters will be stored in a key-value pairing. In specific cases, configuration parameters may be stored as objects. In some cases configurations are stored as JSON or XML and these values will be stored in the same database a BLOB and TEXT values.

The configuration database will maintain application and schema version strings for verifying that the application version of the control system matches the database version. At application startup, the version strings will be checked and compared for equality. Aversion mismatch may result in unpredictable behavior and software crashes due to components attempting to read data values of unrecognized types and formats.

The Configuration Sub-system is designed in a functional manner. Each system component, service, and sub-system knows its configuration. The Configuration Subsystem provides a set of simple methods with a number of overrides that accept a configuration parameter name and its value. The method overrides accept additional parameters that may include type information and other meta data parameters that are needed to properly store parameters.

Configuration changes made in the system are written to audit logs using the Reporting Sub-system. Audit logs must contain the configuration setting, the operator that made the configuration change, and the date/time stamp the configuration setting change was made.

The control system has the capability of storing configuration profiles. Configuration profiles represent a group of configuration settings, which encompass applicator placement, volume of ionized hydrogen peroxide (iHP) to disperse, the number of applicators to activate, and the name of the location an applicator run is being executed.

Configuration profiles are stored using the browser interface and can be saved and loaded on-demand by system operators. The Configuration Sub-system provides remote configuration and calibration access via browser access when the unit is connected to the Internet (6), and by exposing functionality via the API Gateway.

### Device Driver Subsystem

The Device Sub-system manages multiple devices and sensors used by the control system. The Device Driver Sub-system provides operational control and access to the control system's sensor suite and connected applicators.

Application services and sub-systems do not have direct access to the control system's underlying hardware. The Device Driver Sub-system is designed for single threaded access and uses a functional, command mode of operation. The Device Driver Subsystem abstracts objects that represent hardware devices and the drivers needed to control the devices.

Similar to other sub-systems, the driver interface exposes function calls to higher-level software components. These functions are named for the operations they perform on the lower-level hardware. This abstraction is implemented using a low-level command set that sends serial commands over the USB interface to the control board hardware. The command set, is the actual driver that's used to control and measure applicator operation, monitor sensors, and invoke reads on the RFID reader.

The command set provides pin/out operation in which the voltage values on a specific PIN on the control board can be read and supplied back to higher level calling functions. The control system is connected to multiple control boards, which provide general purpose VO. The control board provides the ability to read input voltages from multiple pins. The Device Sub-system will provide the ability of assigning one or more I/O pins for a specific transmission direction and application need. For instance, PinO could be used for flow meter control and measurement, where Pin 16 is used to receive input signals from a SCADA device. The Device Sub-system will expose multiple functions that assign VO operation to a specific pin number on the control board. This provides higher levels of application abstraction with the ability of performing device specific operations without needing to know how to communicate with the underlying hardware. Input read voltage is used for determining flow meter volume. In one embodiment, the input voltage is read between 0 and 5 volts. Input read voltage is used for reading events from proximity sensors. These values will be determined when a proximity sensor has been chosen in the sensor suite.

The Device Sub-system manages communication between the control system software and its underlying hardware resources (applicators, drivers, sensors, etc.). This constitutes a device data pipeline since the Device Sub-system handles the instruction set for multiple discreet sensor devices.

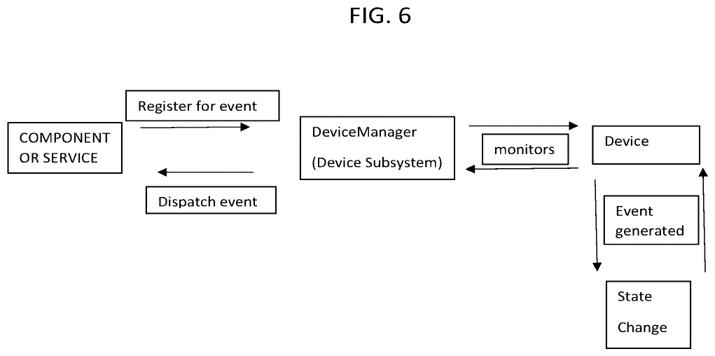

FIG. 6 shows the Device Driver Sub-system event state change design. The sub-systems management component (Device Manager) provides a means of adding, enabling, disabling, and removing devices. Devices may encompass any communicable piece of hardware including applicators. The Device Manager supports monitoring multiple devices in the pipeline through its exposed functions.

The sub-system uses capabilities provided by the Alerts and Events Subsystems to provide the ability for higher level components and services to be notified when a sensor or other hardware device (e.g., applicator) encounters a state change. The Alerts and Events Sub-systems' observer pattern implementation provides this notification capability.

### Events Sub-System

The Event Sub-system supports receiving software and hardware events from a number of sources.

In a particular embodiment, The Events sub-system reuses the Alert Subsystems' registration and publication design and adapt the object model and execution components for the Events Sub-system. Internal components and external software interfaces reuse similar mechanisms as designed by the Alerts Sub-system. The Event Sub-system handles events that can come from hardware and software and dispatches the event to interested sub-systems and components to take action.

Most hardware events are sourced from the control board hardware originating from an applicator code or from one or more sensors attached to control board hardware. Additionally, supervisory control and data acquisition (SCAD A) systems and other external devices may be connected to the control system using one of the available digital or analog pins available on the control board hardware. The system configuration provides the capability of identifying which PIN the hardware device is connected to and the voltage requirements that need to be read from the PIN.

Hardware event detection will be made using a configurable polling method that will check the values on multiple VO pins. When a state change occurs, the producer/consumer mechanism will be used to notify any services or components that are registered for a specific hardware event change. External services and applications must register a RESTful web hook that the control system can call in order to receive hardware event notifications.

The Event Sub-system is also responsible for responding to any shutdown event. A shutdown event can occur internally because of an unrecoverable fault or error, an operator initiated shutdown via browser or API, or because of a hardware event such as an emergency button press or relay shut off. Like all other events, the Event Sub-system handles shutdown events. After the event is handled, the Event Sub-system dispatches events to the System Controller, which is used to gracefully or forcefully terminate the control system.

The Event Sub-system provides the ability to optionally send alerts at specific events. Some use cases may require that an alert be sent when an applicator run begins and ends. Operators can configure the control system to send an alert via email to a specific recipient when the event is triggered via the browser based configuration panel.

### Reporting Subsystem

The Reporting Sub-system is the primary controller for serializing data generated by the control system. Additionally, the Reporting Sub-system is used for generating multiple logs including the system and audit logs.

The Reporting Sub-system stores analytics and execution data using an embedded database. The database used for storing analytics may be the same database used for storing configuration parameters. Additional analytics items may be added as system requirements change. All analytical values will be stored using key-value pairs were suitable and use object storage when necessary. After an applicator run completes, analytical data shall not be changed and is marked as read only.

Applicator run analytics are retrievable at any time and can be used to generate reports. The Reporting Sub-system provides the capability of exporting applicator run analytics in PDF. PDF reports will be rendered so that they are tamper resistant to meet pharmaceutical guidelines. The Reporting Sub-system exposes access to analytics data via the API Gateway. This provides the system with the ability to deliver analytics information via the cloud by retrieving data from the control system units that are connected to the Internet.

The Reporting Sub-system exposes functionality for generating multiple logs through loggers. A standardized logging package is used for generating logs across the system (log4j, Winstone, etc.). In a particular embodiment, the control system will write three logs: a system log that tracks system events and notifications across the system, and an audit log that is used for tracking user driven events and configuration changes, and an execution log that tracks applicator runs and run analytics. Logs are retrieved via the browser interface and can be downloaded as text files for later inspection.

Loggers must write the following items to any configured logs:
Date/Time of the log message
The log text
Sub-system
Log Level (debug, error, info, warning)

### Software Development Kit (SDK)

The SDK (Software Development Kit) provides the ability for technically advanced users, to build applications on top of the control system. The SDK is designed for building applications on top of publicly available services exposed by the control system. Users will determine which services are publicly available and others that are Reserved. Reserved application services are critical to reliable operation of the control system and may cause unpredictable behavior if modified by end users. A possible Reserved service may be the calibration functionality for specific hardware. Users may determine that end users should not have the ability to calibrate hardware, and that functionality should be Reserved for User technicians. Access to the SDK will be facilitated by reusing the API gateway platform.

### Security

Operator security is governed by username and password access to the browser application. The web application uses Role-based Access Controls (RBAC) to limit access to protected configuration settings and controlling specific behavior in the system. The roles and configuration settings and behavior will be determined by the user.

Connection security is possible by using SSL certificates that encrypt communication between the web server and browser. NGINX makes it easy to install SSL certificates providing the ability to secure browser communications. SSL certificates must be installed by SSH-ing into the computer and secure copying the certificates in NGINX's certificate store.

API Key will govern external access to the API Gateway. API keys are unique values generated for software programs to communicate with a web resource. Secure access to the control system will be implemented using API Keys, and a new API key must be generated for each application that requires programmatic access to the control system.

The control system web application utilizes the same API Gateway interface for communication and control. Local security is made by API key and local IP address binding since the web application server is a special application that is granted access. SSL certificates may also be used to secure the communications channel between web browsers and external applications. SSL security will be implemented by using the native capabilities provided by the web server installed on the control system.

### METHOD OF USE

An aspect of the application relates to method of controlling decontamination of a substantially enclosed space, comprising: detecting a micro-organism's presence in a substantially enclosed space, wherein the presence of the micro-organism is sensed by one or more sensors that are present within the substantially enclosed space; alerting a system controller to the presence of the micro-organism in the substantially enclosed space, wherein the system controller is networked to the one or more sensors; informing an operator device of the presence of the micro-organism in the substantially enclosed space, wherein the operator device is networked to the system controller; initiating a decontamination process to remove the presence of the micro-organism in the substantially enclosed space, wherein the decontamination process is applied by one or more applicators networked to the system controller, and further wherein the one or more applicators are present in the substantially enclosed space; and further wherein the system controller initiates the decontamination process by the one or more applicators after the steps of either: (1) ordering the initiation of a decontamination process from the operate device; or (2) ordering the initiation of a decontamination process by an event sub-system, wherein the event sub-system is a non-transitory tangible computer-readable medium comprising instructions to decontaminate a substantially enclosed space.

In certain embodiments, a control system uses a general purpose computer to implement instructions for repeating decontamination cycles of a decontamination apparatus, the instructions comprising: sensing a presence of a pathogen in a substantially enclosed space; communicating the presence of the pathogen to a computer database; identifying the pathogen sensed in the substantially enclosed space using the computer database; selecting a program of decontamination cycles from the computer database based on the identity of the pathogen; communication the selected program to a decontamination apparatus, wherein the decontamination apparatus is networked to automatically follow the program; performing the decontamination cycles according to the program.

Some examples of embodiments using the decontamination apparatus, system, or method of the present disclosure include shipping containers. For example, a shipping container may be equipped with a decontamination system that can sense pathogen load within, or on surfaces of, the container. Exemplary systems can feed information about pathogen load to parties equipped to receive data. In some embodiments, a system can print or record data.

Other examples of embodiments using the decontamination apparatus, system, or method of the present disclosure include import, export, travel quarantine areas or checkpoints. In some embodiments, the system includes a walk-through space or tunnel, conveyer system, moving walkway or any other suitable means for moving persons or objects through the mist generated by the decontamination system.

Still other examples of embodiments using the decontamination apparatus, system, or method of the present disclosure include a vehicle. In some embodiments, the vehicle is a car, truck, bus, train, airplane, or any other form of transportation purposed for the movement of goods or passengers. In further embodiments, the vehicle is an autonomous vehicle.

Yet other examples of embodiments using the decontamination apparatus, system, or method of the present disclosure include space travel, space quarantine, or structures that do not reside on the planet earth.

Some examples of embodiments using the decontamination apparatus, system, or method of the present disclosure include food processing/preparation systems. In some embodiments, the system includes sensors, such as photodetectors, to activate the apparatus. In some embodiments, the system includes sensors for detecting pathogen load.

Still other examples of embodiments using the decontamination apparatus, system, or method of the present disclosure include self-guiding robots networked wirelessly with the customized engineering system. For example, a self-guiding robot equipped with the decontamination system can move around a space or facility, detect contamination via a single or multiple sensors of the same or different types in response to directions received from the customized engineering system. A self-guiding robot equipped with the decontamination system and networked with the customized engineering system can treat a contaminated surface or space until bioload is reduced in a target area.

Yet other examples of embodiments using the decontamination apparatus, system, or method of the present disclosure include emergency biocontamination rapid deployment chambers.

Other examples of embodiments using the decontamination apparatus, system, or method of the present disclosure include farms, ranches, livestock facilities or abattoirs. As non-limiting examples, a decontamination apparatus or system can be installed in a poultry facility, such as chicken coops, or a dairy collection facility.

Still other examples of embodiments using the decontamination apparatus, system, or method of the present disclosure include, but are not limited to, gyms, studios, training facilities, or bathrooms.

Other examples of embodiments using the decontamination apparatus, system, or method of the present disclosure include buildings with a decontamination system integrated into the building systems in order to decontaminate the entire building or specific area of the building. In some embodiments, the system is integrated into new construction. In other embodiments, the system is integrated into the automation or ventilation systems of an existing building. In some embodiments, a decontamination system or apparatus of the present disclosure is programmable or automated.

The present application is further illustrated by the following examples that should not be construed as limiting. The contents of all references, patents, and published patent applications cited throughout this application, as well as the Figures and Tables, are incorporated herein by reference.

### EXAMPLES

### Example 1

### Computer Hardware

The following is an exemplary embodiment of computer specifications:

Computer Hardware Specifications

| | |
|---|---|
| Model | Logic Supply LS-CL100 - 1 × 4 × 32 |
| | Single Board Computer |
| Power | DC 12 V, 36 W |
| CPU Cores | 1 - Intel Braswell Celeron N3160 |
| RAM | 4 GB |
| Disk (SATA-SDD) | 32 GB |
| Display | 2 - HDMI |
| I/O Connectivity | 2 - USB |
| LAN Connectivity | 1 - Gigabit Ethernet |
| Wireless Connectivity | ATHEROS Dual Band Wi-Fi/Bluetooth |
| | 802.11 a/b/g/n |
| Wireless Antenna | 108 mm (4.25″) Wi-Fi Dual Band |
| Operating System | Ubuntu 16.04 LTS x64 |

### Sensor Interface

In an exemplary embodiment, the Arduino MEGA 2560 microcontroller platform is used for interfacing the control system (1) applicator (3) hardware with the computer with the following specifications:

Arduino Mega 2560 Specifications

| | |
|---|---|
| Model | Arduino Mega 2560 Control Board |
| Microcontroller | ATmega2560 - 16 mHz |
| I/O | 54 - Digital I/O Pins |
| | 15 - PWM Outputs |
| | 16 - Analog Inputs |
| | 4 - UARTS |
| I/O Connectivity | 1 - USB |

The SteraMist CES is a fully automated decontamination system that utilizes the facility's existing HVAC system. This involves installing remote SteraMist BIT applicators within the designated space to achieve maximum results. To do this, cabling will be installed throughout that will feed each SteraMist BIT applicator's air, solution, and power. The SteraMist BIT generator and the Programmable Logic Control (PLC) will be housed in a NEMA enclosure in a central location.

This customized engineered system is built for ease of use. Once installed, Service Technicians will determine the ideal specifications to accomplish maximum efficacy. This will be programmed into the PLC and will interface with the building's HVAC system for room isolation and aeration. Programming will allow for multiple cycles to be retained. Programming will also allow for continued cycle when multiple pods fail and will redirect the remaining dose to the remaining applicators rather than shut off. The entire system can be developed for multiple rooms and various specifications; and controlled remotely through the HMI interface.

Once the area has reached its specified criteria for disinfection/decontamination, the integrated Drager monitoring system will test the air at required intervals until the H2O2 concentration is below Ippm. The status of the cycle is monitored with remote indicators and can be integrated into a SCADA monitoring system.

### General Specifications:

### Ceiling Mounted Applicators:

Mounting boxes and cover plates to be fabricated of 14 gage stainless steel.

Mounting boxes will have pre-cut and installed bulkhead fittings and glands.

Applicator cover plates equipped with warning buzzer.

Applicator clean dry air (CDA) is 2 SCFM per applicator at 90-100 psi and to be provided by customer.

Three (3) different color alert status lights (Red/Warning, Yellow/Injection, Dwell, Aeration, and Green/All Clear to Enter Space and Flashing Red/Alert for a Fault—can be included with each applicator box.

Standardized SS mounting box to be used for either fixed or oscillating configurations. Back box dimensions are 11″ W×9″ W×6″ D, with 1.5″ W lip around entire back box opening.

Ceiling mounted back boxes will have precut and installed bulkhead fittings and glands to make installation easier.

Ceiling mounted back boxes can be installed no more than 100 ft (30 m) maximum distance from the control cabinet, with no more than 12-15 ft total elevation change.

Applicator cover plates have dimension measuring 14.5″ L×12.5″ W×0.875″ D and option to be equipped with audible buzzer and colored lamps (red, yellow, green standard).

Optional oscillating applicator function achieved via a direct drive stepper motor and will be adjustable in oscillation speed and angle via controls on the HMI.

### Control Panel:

Houses fluid system, air system, and electronic controls.

Panel comprised of a single enclosure.

Control cabinet compressed air supply will be supplied by the facility (CDA at 100 psi, 4 SCFM min).

HMI Screen depicted below and included in the quote is 15″, but HMI screen size options also includes 7″, 9″, 12″, and 19″.

E-Stop Button placed inside decon room for emergency termination of any sequence.

E-Stop Button depicted in the below image under the HMI screen.

Fluid Pumping & Priming Panel:

Wall mounted enclosure with dimensions measuring 24″ H×24″ W×12″ D.

Pumping station can utilize 55- or 10-gallon drum with a suction tube assembly.

Spill containment structure that allows up to 60 gallons of BIT solution to be controlled during a spill, Customer may install a localized drain and hose at their chosen location.

Pumping controls with signaling to Control Panel for low-fluid indication.

Station will NOT have fluid calibration capabilities.

### RFID Capabilities for BIT Solution Container

Remote pumping station capabilities available, allows for main control cabinet to be in a separate location that BIT solution.

All applicator priming return lines will be plumbed to return BIT solution to the drum.

BIT solution container will reside on a scale which will accurately measure the solution consumed aiding in dosing accuracy and tracking of solution used in each spray cycle.

Customer Programmed HMI with the following capabilities:

Up to 20 individual user IDs with password protection. IDs can be setup and changed through an administrative user.

Dual “Dry Contact” VO for interface with HVAC system and door interlock controls (2 inputs and 2 outputs). Adjustable dwell-time setting for HVAC control. Built in logic for door interlock sequencing and cross contamination prevention.

Ability to start and stop cycles based on “clock times” as well as manual start/stop.

PDF Reports for each cycle pushed to USB memory stick, network location, or emailed directly from the system itself.

Fault monitoring includes fluid flow, air flow and arc detection.

The maximum distance the fluid priming skid should run to the control panel is 328 ft with combined rise and run lengths and no more than 20 ft rise.

The maximum distance the control panel should run from the applicator is 98 ft with combined rise and run, and no more than a 10 ft rise.

Location of Drager monitor to be placed on the clean side to monitor PPM level from the HMI, remote sensor mounted into the exhaust duct. If individual exhaust exists for the three areas being disinfection, then three monitors will be required, one for each duct. Drager monitor(s) with remote mounted duct sensor (total depends on the HVAC system design)

Customer provides 120V AC with a 20 amp breaker at the source, final connection is to be provided by other party. Company will provide airtight dampers.

### Standard System Performance & Install Specifications

Utility requirements:
120 VAC, 10, 15 amp.
Clean Dry Air, 2 SCFM @90-100 psi (per applicator).
25 mL/min fluid flow rate.
30 psi applicator regulated air.

‘A” OD poly tubing lines for Fluid and Air supply, connecting control panel and applicators, and applicator fluid return lines to pumping cabinet.

Air and Fluid supply lines cannot exceed 100 ft (30 m) maximum distance from the control cabinet, with no more than 12-15 ft total elevation change. (This includes rise and run lengths combined).

Fluid return lines cannot exceed 100 ft (30 m) maximum distance from the control cabinet, with no more than 12-15 ft total elevation change. (This includes rise and run lengths combined).

⅜″ OD poly tubing lines connecting fluid pump cabinet to control cabinet (for BIT fluid supply from gallon drum to internal control cabinet Aux. reservoir).

Fluid supply line cannot exceed 300 ft (92 m) maximum distance from the control cabinet, with no more than 20 ft total elevation change. (This includes rise and run lengths combined).

CAT5e wire connecting baseline (master) control panel to subsequent slave control panels (for applicator 5+) cannot exceed 300 ft.

Additional “slave” control panels will have the same restrictions for air/fluid supply line distances as well as fluid supply line distances from pumping cabinet as mentioned above.

Each applicator will have a total 18 wires (18 gauge, multicolor) placed between ceiling back box and control cabinet and 4 of the 18 will need to be wired in a separate independent wire core.

Fluid pumping and priming cabinet will have a total of 7 wires (18 gauge, multicolor) and the RFID reader cable, all will be wired between the control cabinet and pumping cabinet.

### Injection:

Customer initiates the start of the cycle—system delays the start of the system for the HVAC to be shutdown (either manually or by signal sent from system).

The systems monitor the amount of solution requested both in flow rate during injection, and total amount delivered (system will fault if the system detects a loss of flow from any applicator—if more than one applicator is installed in a treatment area the system can be programed either to shut the cycle or allow the remaining applicator(s) to continue until the correct required amount of solution has been delivered).

Air *CDA* is also monitored during the injection cycle for a lost at each applicator and at the inlet of the air supply.

The arc is also monitored at each applicator.

### Dwell:

Dwell will commence after the injection cycle (this pause period is timed and is adjustable by customer).

During the dwell cycle the system is in standby, treatment area(s) remain under the control of the system.

### Aeration:

After dwell, aeration will commence. The system will signal the HVAC/exhaust system to turn on (if equipped).

The system will monitor the treatment area(s) exhaust for residual concentrations of solution (all doors remain locked until a preset safe level is achieved) *if equipped* or the system can be set to release door(s) upon a preset time.

### Cycle Completion:

Upon a completed cycle system can send information to customers server for distribution as customer deems necessary (if equipped).

Upon a faulted cycle system can send information to customers server for distribution as customer deems necessary (if equipped).

### Example 2

### Customized Engineering System

Effective disinfection by activated hydroxyl ions as used in the present application depends on the surface area of the droplets that are applied to surfaces; that is the smaller the droplets, the greater the surface area of activated hydroxyl ions on the total cloud of droplets, and thus, the more effective the disinfection method. In fact, soaking a surface for disinfection undermines effectiveness of activated hydroxyl ions because once a surface is soaked the activated ions will not be brought into contact with the bacteria for disinfection.

Activation of the cleaning fluid to produce activated hydroxyl ions may occur through passage of the fluid, for example, an electric arc current, an electromagnetic field, or photonic energy. The fluid may be generated as a spray via, for example, nebulization, ultrasonices, pneumatic spray, or mechanical pressure. However, blowers are not used in the method of the application to generate a spray, as a blower will generate a powerful stream of large droplets that will soak a surface with fluid, which both undermines the impact of any activated hydroxyl ions.

The methods of the application require that a very dry mist (very low diameter aerosol particles as described herein) be generated which carries activated hydroxyl ions through a space to a surface for decontamination. The activated hydroxyl ions make contact with pathogens before recombining to form harmless diatomic oxygen and water (it is an advantage of the approach herein that no chemical residue remains on the disinfected surface). Preferred embodiments of the present application use, for example, a cleaning fluid that comprises 0.3% to 9% hydrogen peroxide as a source of an active species for decontamination of an article or substantially enclosed space. Preferred aerosol droplets that carry activated hydroxyl ions are 0.3-1.0 microns in diameter, with most preferred to average 0.7 microns in diameter. Accordingly, any automated systems applying the present methods require exacting parameters for performance.

Methods and technologies preferable for use in decontamination processes are discussed in U.S. Pat. No. 10,391,188, which is incorporated herein by reference. A decontamination fluid mist is activated to produce an activated decontamination fluid mist. The activation produces activated species of the decontamination fluid material in the mist, such as the decontamination fluid material in the ionized, plasma, or free radical states. At least a portion of the activatable species is activated, and in some cases some of the promoting species, if any, is activated. A high yield of activated species is desired to improve the efficiency of the decontamination process, but it is not necessary that all or even a majority of the activatable species achieve the activated state. Any operable activator may be used. The activator field or beam may be electrical or photonic. Examples include an AC electric field, an AC arc, a DC electric field, a DC arc, an electron beam, an ion beam, a microwave beam, a radio frequency beam, and an ultraviolet light beam produced by a laser or other source. The activator causes at least some of the activatable species of the decontamination fluid in the decontamination fluid mist to be excited to the ion, plasma, or free radical state, thereby achieving “activation”. These activated species enter redox reactions with the cell walls of the microbiological organisms, thereby destroying the cells or at least preventing their multiplication and growth. In the case of the preferred hydrogen peroxide, at least some of the H2O2 molecules dissociate to produce hydroxyl (OH—) and monatomic oxygen (O—) ionic activated species. These activated species remain dissociated for a period of time, typically several seconds or longer, during which they attack and destroy the biological microorganisms. The activator is preferably tunable as to the frequency, waveform, amplitude, or other properties of the activation field or beam, so that it may be optimized for achieving a maximum recombination time for action against the biological microorganisms. In the case of hydrogen peroxide, the dissociated activated species recombine to form diatomic oxygen and water, harmless molecules.

Exemplary decontamination devices/systems of the present disclosure comprise an applicator having a cold plasma arc that splits a hydrogen peroxide-based solution into reactive oxygen species, including hydroxyl radicals, that seek, kill, and render pathogens inactive. The activated particles generated by the applicator kill or inactivate a broad spectrum of pathogens and are safe for sensitive equipment. In general, decontamination devices/systems of the present disclosure allow the effective treatment of an exemplary space measuring 104 m2 in about 75 minutes, including application time, contact time, and aeration time. Decontamination devices/systems of the present disclosure are scalable and configurable to be effective in any size or volume of space/room/chamber/container. The scalability may be accomplished by the size of the device, by the manual control of the decontamination fluid, or by programming the air pressure of the device and the consequent fluid flow rate as a function of the input space/room/chamber/container parameters.

Conventional methods of decontamination are less effective in decontaminating closed spaces. This application discloses that decontamination using a very dry mist comprising ionized hydrogen peroxide provides unexpectedly high levels of kill rate of pathogens (which encompasses bacteria, fungi, protozoan or viruses), such as, e.g., Candida auris, in small enclosures, semi-enclosed spaces and closed areas (a small enclosure is an area of 12″×12″×12″ or less; a semi-enclosed space is an area in which part of a small enclosure is open to other areas; a closed area is an area in which no parts of the small enclosure are open to other areas).

A very dry mist is a mist in which particles have particle size diameter within the ranges of about 0.1-0.2 microns, 0.1-0.3 microns, 0.1-0.4 microns, 0.1-0.5 microns, 0.1-0.6 microns, 0.1-0.7 microns, 0.1-0.8 microns, 0.1-0.9 microns, 0.1-1 microns, 1-1.1 microns, 1-1.2 microns, 1-1.3 microns, 1-1.4 microns, 1-1.5 microns, 1-1.6 microns, 1-1.7 microns, 1-1.8 microns, 1-1.9 microns, 1-2 microns, 0.5-0.6 microns, 0.5-0.7 microns, 0.5-0.8 microns, 0.5-0.9 microns, 0.5-1 microns, 0.5-1.1 microns, 0.5-1.2 microns, 0.5-1.3 microns, 0.5-1.4 microns, 0.5-1.6 microns, 0.5-1.7 microns, 0.5-1.8 microns, 0.5-1.9 microns, 0.5-2 microns, 0.5-2.1 microns, 0.5-2.2 microns, 0.5-2.3 microns, 0.5-2.4 microns, 0.5-2.5 microns, 0.5-2.6 microns, 0.5-2.7 microns, 0.5-2.8 microns, 0.5-2.9 microns, 0.5-3 microns, 0.5-3.1 microns, 0.5-3.2 microns, 0.5-3.3 microns, 0.5-3.4 microns, or 0.5-3.5 microns. In certain embodiments, the very dry mist has particles with particle diameter size in the range of about 0.5-3 microns, preferably on average 0.7 microns.

In certain embodiments, the customized engineering system described herein monitors the size of the aerosol droplets being produced, so that the aerosol droplets carrying activated hydroxyl ions form a very dry mist as described herein. In preferred embodiments, the population of aerosol droplets at least 80%, 90%, 95%, 100% are within the size range of 0.3-1.0 microns in diameter. In particular embodiments, the size of aerosol droplets is monitored by use of laser scanning of aerosol droplet size. Optical measurements may be performed with a sensor or a particle detector placed in the detection zone after the point of activation of hydroxyl ions on the aerosol droplets, sensors may be an optical particle counter (OPC), a laser particle counter (LPC), or a condensation particle counter (CPC). OPCs or LPCs can detect particle sizes larger than 0.1 microns. The customized engineering system is equipped with a computer processor as described herein, which receives data regarding the size range of aerosol droplets carrying activated hydroxyl ions. The customized engineering system is programmed to adjust control parameters governing the size of particles in the very dry mist to maintain the population of aerosol droplet sizes within the desired range.

The customized engineering system includes a programming clock, and provides air pressure control and fluid flow control through use of one or more potentiometers. The programming clock provides the ability to automate cycles of decontamination within a small enclosure. The cycles of decontamination controlled by the programming clock may, for example, include cycles of spraying a very dry mist for thirty seconds, stopping spray for ten seconds, and then re-starting spraying for another thirty seconds, etc. repeating such cycles for a fixed period of time. The programming clock can be set manually by a user or controlled remotely by wireless by the user or a computer processor with pre-programmed decontamination cycles that are transmitted to the device for deployment.

In certain embodiments, the time period during sprayings may be 10-1800 seconds, 10-1200 seconds, 10-900 seconds, 10-600 seconds, 10-300 seconds, 10-180 seconds, 10-150 seconds, 10-120 seconds, 10-90 seconds, 10-60 seconds, 10-45 seconds, 10-30 seconds, 30-1800 seconds, 30-1200 seconds, 30-900 seconds, 30-600 seconds, 30-300 seconds, 30-180 seconds, 30-150 seconds, 30-120 seconds, 30-90 seconds, 30-60 seconds, 30-45 seconds, 60-1800 seconds, 60-1200 seconds, 60-900 seconds, 60-600 seconds, 60-300 seconds, 60-180 seconds, 60-150 seconds, 60-120 seconds, 60-90 seconds, 90-1800 seconds, 90-1200 seconds, 90-900 seconds, 90-600 seconds, 90-300 seconds, 90-180 seconds, 90-150 seconds, 90-120 seconds, 120-1800 seconds, 120-1200 seconds, 120-900 seconds, 120-600 seconds, 120-300 seconds, 120-180 seconds, 120-150 seconds, 150-1800 seconds, 150-1200 seconds, 150-900 seconds, 150-600 seconds, 150-300 seconds, 150-180 seconds, 180-1800 seconds, 180-1200 seconds, 180-900 seconds, 180-600 seconds, 180-300 seconds, 300-1800 seconds, 300-1200 seconds, 300-900 seconds, 300-600 seconds, 600-1800 seconds, 600-1200 seconds, 600-900 seconds, 900-1800 seconds, 900-1200 seconds or 1200-1800 seconds.

In certain embodiments, the time period between two consequent sprayings may be 1-600 seconds, 1-300 seconds, 1-180 seconds, 1-150 seconds, 1-120 seconds, 1-90 seconds, 1-60 seconds, 1-45 seconds, 1-30 seconds, 1-15 seconds, 10-600 seconds, 10-300 seconds, 10-180 seconds, 10-150 seconds, 10-120 seconds, 10-90 seconds, 10-60 seconds, 10-45 seconds, 10-30 seconds, 30-600 seconds, 30-300 seconds, 30-180 seconds, 30-150 seconds, 30-120 seconds, 30-90 seconds, 30-60 seconds, 30-45 seconds, 60-600 seconds, 60-300 seconds, 60-180 seconds, 60-150 seconds, 60-120 seconds, 60-90 seconds, 90-600 seconds, 90-300 seconds, 90-180 seconds, 90-150 seconds, 90-120 seconds, 120-600 seconds, 120-300 seconds, 120-180 seconds, 120-150 seconds, 150-600 seconds, 150-300 seconds, 150-180 seconds, 180-600 seconds, 180-300 seconds, or 300-600 seconds. In one example, the time period between two consequent sprayings is 60 seconds.

In some cases, the time period during spraying is 90 seconds, with 60 second intervals between spraying. In some embodiments, a spray circle comprises a spray time and a break time, and a complete decontamination process comprises 1, 2, 3, 4, 5, 6, 7, 8, 9 or 10 spray circles. (Spray circle=spray+interval−total number of circles.)

In certain embodiments, the customized engineering system will possess a computer processor that can calculate the appropriate settings (e.g., flow rate, air pressure, number and length of decontamination cycles) to produce a very dry mist comprising ionized hydrogen peroxide that will effectively decontaminate a closed space. In such embodiments, the user may enter the parameters of the small enclosure manually to the device, or enter them remotely by a wireless connection. The operation of the system can be fully automated, fully remotely controlled, or may be semi-automated (e.g., uses cycles of decontamination performed automatically according to parameters that have been manually entered).

It is a common problem of the conventional technology that excessive air pressure reduction produces mist particles that are too large to achieve a desired mist/fog profile. At the same time, particularly enclosed spaces often require significant air pressure reduction. These opposing constraints of a decontamination system are addressed by certain embodiments of the present disclosure. Namely, by programming the processor to control the potentiometer based on the input parameters of the small enclosure, a user can regulate a fluid flow rate in synchronization with the air pressure. As a result, reducing the fluid flow rate while simultaneously lowering the air pressure maintains the mist/fog particle size small, while limiting the distance the spray can reach. In this manner, the mist sprayed by the customized engineering system remains within the boundaries of the enclosed space, without creating excessively wet and dense fog. The programmable balance between the air pressure and the fluid flow rate, therefore, prevents saturating surfaces opposite to mist applicators, increased moisture accumulation due to condensation, false negative validation results or increased aeration times of the enclosure.

In some embodiments, the customized engineering system may include or may be configured to access a database listing room characteristics in which the customized engineering system is deployed. In addition or alternatively, the customized engineering system may include a system for collecting and/or generating data regarding characteristics of a room in which the customized engineering system is deployed. In such cases, any system known in the art for collecting, generating and/or analyzing characteristics of a room may be used, depending on the data to be generated. Examples include spatial sensors, photo recognition systems and/or dosimeters. A system may, in some embodiments, be operationally coupled to a CPU. Alternatively, a CPU may be configured to access room characteristic data from a database. In either case, CPU may be configured to retrieve and access data regarding characteristics of the room in which the customized engineering system is deployed and determine an operating parameter of the applicator for application of ionized hydrogen peroxide, such as a position of the applicator based on the data. In some embodiments, the determined operating parameter may be relayed via a user interface such that a user of the customized engineering system may be informed to invoke the operating parameter for the customized engineering system. In other cases, a CPU may be configured to send a command in accordance with the determined operating parameter to a means within the customized engineering system for automatically invoking the operating parameter, such as automatically moving the direction of spray of the applicator.

In some embodiments, a system may be used to measure doses of ionized hydrogen peroxide received at an object or spot in a room in which the customized engineering system is deployed. In particular, measuring the dose of ionized hydrogen peroxide at an object or spot in a room may aid in determining operating parameter of the applicator, such as optimizing the placement of the applicator. As noted above, one of the primary factors affecting ionized hydrogen peroxide effectiveness on an object is distance to the object. Through the operational coupling of system to a CPU, the CPU may be configured to retrieve measurements from the system, determine an operating parameter of the applicator based on the measurements, such as a position of the applicator, and either relay the determined operating parameter to a user interface and/or send a command in accordance with the determined operating parameter to a means within the customized engineering system for automatically invoking the operating parameter, such as an applicator. In general, any system known in the art for measuring spray doses may be used for the system.

The customized engineering system may include or may be configured to access a database listing characteristics of one or more rooms and/or apparatus may include a system for collecting and/or generating data regarding characteristics of a room. Any system known in the art for generating, collecting and/or analyzing characteristics of a room may be used. Examples include dosimeters, spatial sensors and/or photo recognition systems. In some cases, apparatus may further include a CPU to retrieve data, determine a position of the applicator based on the data, and either relay the determined position to a user interface and/or send a command in accordance with the determined position to a means within the customized engineering system for automatically moving the applicator.

In certain embodiments, the customized engineering system comprises laser diffraction technology to quantify particle size distribution in in the spray. The applicator is mounted outside in ambient air and the spray travels through an enclosed nozzle. The spray is formed by ultrasonics, or similar means, generated within a chamber within the customized engineering system and the spray flows through the applicator to the outer air. Midway through the nozzle, the spray travels through the zone in which the laser is projected thus causing the laser beam to diffract after colliding with the particles. A collection of sensors across from the laser source measure these diffraction patterns and, using Mie theory (an analytical solution of equations for the scattering of electromagnetic radiation by spherical particles), interprets them to quantify particle size distribution of the ionized particles in the spray. Since the method herein relies on application of a very dry mist, the customized engineering system may be programmed to adjust air valve, or other operating parameters, to maintain suitably low mean diameter of ionized particles as determined by laser diffraction technology.

In certain embodiments, the customized engineering system will automatically launch and execute a mission, for example, a cleaning mission, if the customized engineering system is ready. In certain embodiments, the customized engineering system comprises motion sensors that can detect a presence of a human being in the area. The customized engineering system is equipped with warning mechanisms, such as an alarm to sound and/or flashing lights if a human is detected in an area of operation. The customized engineering system will automatically drive away from a human being, quiet itself, or turn off its power supply to quiet the customized engineering system when human being is present. The customized engineering system therefore behaves in a smart way by interrupting its mission so that the human being is not affected by spray. The decontamination customized engineering system monitors the movement of humans within its operating area and, in embodiments, monitors for the leaving of humans from the area, including a time delay before re-activating. When the human has left the area, the customized engineering system autonomously returns to where it discontinued its mission and completes coverage of that room. The customized engineering system, therefore, completes its mission throughout the space while accommodating for the presence of humans within that space.

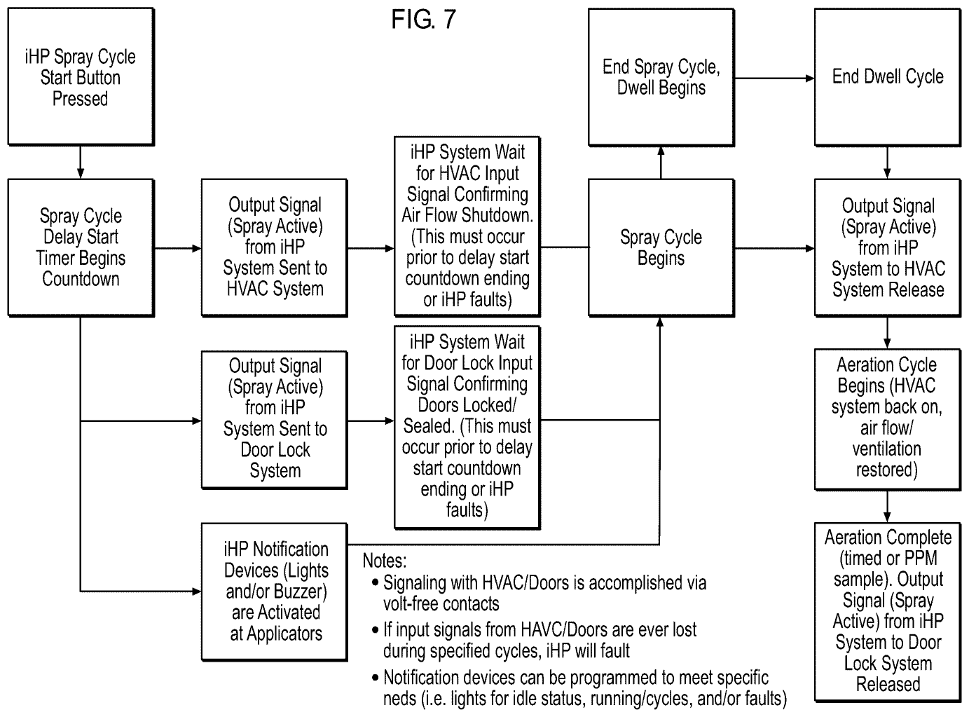

An embodiment of the customized engineering system is shown in FIG. 7. The user initiates the start of the cycle—system delays the start of the system for the HVAC to be shutdown (either manually or by signal sent from system). As can be seen in FIG. 7, the ionized hydrogen peroxide (iHP) spray cycle begins when the start button is pressed (this may be done through the networked systems described herein via a user interface).

Following pressing the start button, a spray cycle delay start timer begins its countdown. This enables any individuals in areas to vacate them before the decontamination begins. It also gives time for individuals to leave before doors are locked so that areas for decontamination are sealed. The system may trigger warning alarms that make alarm sounds to alert individuals in the areas targeted for decontamination and/or verbal warnings that are heard within areas for decontamination, including, for example, a verbal countdown.

Notification devices, such as lights or buzzers, may be present on applicators which are located within areas for decontamination; these notification devices will start flashing or buzzing to alert individuals within the areas to leave. An output signal is sent from the customized engineering system to the building HVAC system to alert the HVAC system that the spray is active; however, the ionized hydrogen peroxide spray will not begin at this point, instead the system will wait for the HVAC system to signal the air flow has shutdown in the targeted areas for decontamination. The delay start countdown does not end until after the customized engineering system has received an input signal from the HVAC system that air flow has shutdown. An output signal is also sent from the customized engineering system to the building door lock system; the system waits for a door lock input signal that confirms that all doors are locked before ending the delay start countdown.

The spray cycle begins once the system has received notification that all doors have been sealed and airflow has been shutdown. The customized engineered system monitors the amount of solution requested both in flow rate during injection, and total amount delivered (system will fault if the system detects a loss of flow from any applicator—if more than one applicator is installed in a treatment area the system can be programed either to shut the cycle or allow the remaining applicator(s) to continue until the correct required amount of solution has been delivered).

The spray cycle ends based on the programmed parameters, and then the dwell cycle begins. Dwell commences after the injection cycle (this pause period is timed and is adjustable by customer). During the dwell cycle the customized engineered system is in standby, treatment area(s) remain under the control of the customized engineered system.

Once the dwell cycle ends, an output signal is sent from the customized engineering system to the HVAC system release. After dwell, aeration commences. The customized engineered system will signal the HVAC/exhaust system to turn on (if equipped).

After the HVAC system is released an aeration cycle begins in which the HVAC system is back on and air flow/ventilation restored. The aeration cycle proceeds to remove the residue of the ionized hydrogen peroxide decontamination (diatomic oxygen and water as described herein) from the room on either a timed based is based on measuring parts per million (PPM) air samples (or both). Once the aeration cycle is complete, an output signal is sent from the customized engineering system to the door lock system to be released. The system monitors the treatment area(s) exhaust for residual concentrations of solution (all doors remain locked until a preset safe level is achieved) or the system can be set to release door(s) upon a preset time.

The system may be equipped with cameras and the ability to view through the cameras the areas that are to be decontaminated; the camera views may either be assessed by a user or by an algorithm designed to recognize human or animal movement in the area for decontamination, if the algorithm recognizes such movement that the spray cycle will fault until the issues is resolved.

Air CDA is also monitored during the injection cycle for a lost at each applicator and at the inlet of the air supply. The arc is also monitored at each applicator. An air monitor is placed in appropriate locations to view monitoring of PPM level. If each area being decontaminated has a separate exhaust and has the possibility of being operated individually then individual low-level monitors can be used for each treatment area. The monitors will monitor the peak PPM at the start of the aeration cycle to provide a data set point (providing a range from maximum to minimum PPM levels).

Upon a completed cycle system can send information to the users server for distribution as user deems necessary. Upon a faulted cycle system can send information to users server for distribution as user deems necessary.

In a certain embodiments, the customized engineering system is used to fog sealed rooms in healthcare, industrial, commercial and institutional settings. The use rate to achieve a minimum fogging concentration of ionized hydrogen peroxide is 0.5 ml per cubic foot of enclosure (room) volume). The software will calculate the dose based on volume entered, and measure that the correct dose is dispensed through the applicators. The application time needed to achieve the required concentration will vary depending upon the room size and number of applicators. Once the minimum fogging concentration is achieved, dwell time must be maintained for a minimum of 15 minutes before initiating aeration of the room. In particular embodiments, the system will begin with these parameters (dose rate/contact time) preset. In certain embodiments, where the system can assess the changing environment (e.g., presence of large scale equipment), the system may have the capability to adjust. The room is aerated until the room reads as under 1 part per million (PPM) of hydrogen peroxide as measured by standard air monitors. Once the room has been appropriately aerated, the room can be entered without personal protective equipment and returned to service. Aeration will naturally occur without mechanical assistance when time is not a factor. Venting to the outside of the building, air scrubbers and fans can all be used after contact to speed the aeration process. Dehumidifiers, fans or HEPA filters with carbon filters are also effective in removing airborne hydrogen peroxide. As ionized hydrogen peroxide breaks down into humidity and oxygen, there is no need to wipe and there are no residues left after treatment.

### Example 3

### Immune Building

The customized engineering system is fully automated. The system is integrated with a facility's HVAC system. Once installed, the system can either rely on preset parameters or specifications that are determined by the system user or technical consultants. In certain embodiments, the system includes a programmable logic control that is housed in a central location. In certain embodiments, the system is controlled by an algorithm that uses machine learning and preset parameters to determine the control and performance of the decontamination system. A machine learning neural network is trained to identify the differences between the stages of the spray cycle, the aeration cycle, and the environmental responses and input parameters described herein. The neural network then acts as an artificial intelligence to control the decontamination system described herein based on environmental inputs due to different sensor signals regarding the presence of pathogens within the building. Users may assume control of the customized engineering system from the artificial intelligence, and either operate through manual commands delivered through the user interface, or through re-programming preferred preset parameters. The use of an artificial intelligence to control the installed and fully automated customized engineering system to perform decontamination cycles as described herein creates an immune building. The customized engineering system functions as an immune system that will respond automatically to detected threats by initiating decontamination in specific contaminated areas of the building as described herein. Based on the identification of particular biological hazards, different spray cycles and dwell cycles can be initiated as appropriate to eliminating the identified biological hazard. Programming allows for multiple cycles to be retained by the system and accessed for deployment. Programming also allows for continued cycles when or if multiple pods fail and redirect the remaining does to the remaining applicators rather than shut off. The system can be used in multiple rooms and for various specifications; and remotely controlled through user interfaces as described herein.

In a particular embodiment, the immune building system is used for the decontamination and sterilization of the specified area. The immune building must be able to sterilize to total volume of the specified area. The immune building system comprises at least one decontamination agent container, decontamination agent distribution piping, decontamination agent pumps, decontamination heads (applicators), and a central control system. The number and location of decontamination heads varies with location requirements. The immune building system begins when the operator (which may be the artificial intelligence, or optionally a manual user who takes control) starts the decontamination process. The system confirms the system status including, but not limited to, available quantity of hydrogen peroxide (optional), hydrogen peroxide expiry date and system readiness for operation. The system undergoes signal exchange with the networked HVAC system; the HVAC system is ordered to stop and the immune building system receives confirmation a stop has occurred. Injection of decontamination solution as a spray mist as described herein is initiated; ionized hydrogen peroxide injection occurs up to a defined quantity based on cubic volume. Spray is concentrated on the worst case location for decontamination; confirmation of decontamination solution consumption (per head) is monitored and assessed by the system. The system gives time for contact to occur with the biological hazard and the sprayed ionized hydrogen peroxide mist. Signal exchange then occurs to restart systems that were stopped to perform the decontamination cycle. The system gives time to allow the flushing with fresh air until ionized hydrogen peroxide concentration has been reached under 1 PPM. In particular, confirmation of safety concentrations is monitored at the worst case locations for biological hazard.

The immune building system is integrated with the HVAC system so as to be able to exchange signals with the HVAC control system. The system can send stop HVAC and restart HVAC signals. The system will be able to receive from the HVAC control system a confirmation of HVAC system stop or HVAC system restart in reply to the given signals from the immune building system. The control system shall allow an integration with optional equipment; the system will be able to send start, monitor, and restart signals. The system will be able to receive from the optional equipment filling lines control system a confirmation of system Stop or System restart in reply to the Stop and Restart signals. The immune building system is prepared to run fully automatic from the moment the process is started to the moment the process is finished including the generation of a process report. The immune building system is design to guarantee that the sterilized rooms and materials have a residual of under 1 ppm ionized hydrogen peroxide as measured at the cycle end.

The immune building system enables adjustment of at least the following parameters: ionized hydrogen peroxide injection rate (per head), ionized hydrogen peroxide injection time, ionized hydrogen peroxide setpoint volume (concentration), ionized hydrogen peroxide contact time, aeration time, and aeration setpoint value (concentration) (optional). In case of temporary interruption of the ionized hydrogen peroxide supply during the sterilization cycle, the cycle is aborted and an alarm is triggered. It is possible to stop the sterilization cycle at any time, by manual override if need be. A lockable emergency stop button is provided for manual override to halt the decontamination in progress. Visible indications such as, system in operation (yellow), system in alarm (red), system ready (green), may be installed or adjusted according to user needs. All ionized hydrogen peroxide equipment has one working hours counter and one cycle counter each. The system can control the correct flow of each individual head. The system may conduct a purge of all lines prior to the beginning of new cycle into a waste container.

Change of set parameters can be made from a central operator interface. These changes can be made by authorized access profile. Recipe configuration shall be possible by authorized access profile. The operator is able to notice, to check and to acknowledge the relevant alarms on the operating panel. A machine error message will be released in case of malfunction between programming logic control and controllers. The system has an interface to an onsite IT network using Ethernet connection. The system is equipped with an Industrial PC to allow operator interface, data entry, data storage and connection with programming logic control and controllers. The recorder has the possibility to record, save, archive and retrieve critical data of the process cycle. System will register, produce and issue data, which include different states of process and equipment, measurements, alarms and any other relevant information.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. A multi-configuration system for decontamination comprising:
a general-purpose computer;
a sensor package;
one or more control boards;
one or more applicators; and
an operator device;
wherein the general-purpose computer is networked by one or more control board(s) to the sensor package, wherein the sensor package can detect the presence of micro-organisms; and
the sensor package is networked to the one or more applicators, wherein the one or more applicators are configured to apply a decontamination process to remove micro-organisms; and
the operator device is networked to the general-purpose computer via an application programming interface (API) gateway, wherein the operator device displays a network interface to an operator; and
the API gateway provides access to a system controller.

2. The system of claim 1, wherein the system controller comprises a set of sub-systems, and wherein the set of sub-systems are linked by a bi-directional interface to the system controller; and
the set of sub-systems comprises:
an alerts sub-system, wherein when the sensor package detects the presence of a micro-organism and then the alerts sub-system alerts the operator by a display on the network interface; and
a device driver sub-system, wherein the device driver sub-system networks by a uni-directional interface to the sensor package and the applicators.

3. The system of claim 1, wherein the system is manually controlled by one or more individuals via the operator device.

4. The system of claim 1, wherein the system is under event driven control by the system controller, wherein the system controller receives an alert to the presence of micro-organisms.

5. The system of claim 1, wherein the system is under remote control by one or more individuals via the operator device.

6. The system of claim 1, wherein the applicators begin a decontamination cycle when instructions are received from the system controller via the device driver sub-system.

7. The system of claim 2, wherein the set of sub-systems further comprises an events sub-system.

8. The system of claim 2, wherein the set of sub-systems further comprises a reporting sub-system.

9. The system of claim 2, wherein the set of sub-systems further comprises a configuration sub-system.

10. The system of claim 2, wherein the set of sub-systems further comprises a software development kit.

11. The system of claim 1, wherein the sensor package comprises one or more control boards networked to sensors and networked to the general-purpose computer.

12. The system of claim 1, wherein the sensors are one or more selected from the group comprising photosensors, voltaic sensors, weight sensors, moisture sensors, and pressure sensors.

13. The system of claim 1, wherein the general-purpose computer comprises a single computer control board.

14. A non-transitory, tangible computer-readable medium comprising instructions to decontaminate a substantially enclosed space, comprising a routine of set instructions for causing a system of claim 1 to perform the steps of:
detecting a micro-organism's presence in a substantially enclosed space, wherein the presence of the micro-organism is sensed by one or more sensors that are present within the substantially enclosed space;
alerting a system controller to the presence of the micro-organism in the substantially enclosed space, wherein the system controller is networked to the one or more sensors;
informing an operator device of the presence of the micro-organism in the substantially enclosed space, wherein the operator device is networked to the system controller;
initiating a decontamination process to remove the presence of the micro-organism in the substantially enclosed space, wherein the decontamination process is applied by one or more applicators networked to the system controller, and further wherein the one or more applicators are present in the substantially enclosed space; and
further wherein the system controller initiates the decontamination process by the one or more applicators after the step of
ordering the initiation of a decontamination process by an event sub-system, wherein the event sub-system is a non-transitory tangible computer-readable medium comprising
a set of instructions for causing the one or more applicators to initiate the decontamination process after the one or more sensors have detected the presence of a specific micro-organism.

15. The non-transitory computer readable medium of claim 14, wherein the specific micro-organism is a pathogen.

16. The non-transitory computer readable medium of claim 16, wherein the pathogen is a targeted bioterror agent.

17. The non-transitory computer readable medium of claim 17, wherein the targeted bioterror agent is selected from the group consisting of anthrax (Bacillus antracis), plague (Yersinia pestis), and tularemia (Franciscella tularensis).

18. The non-transitory computer readable medium of claim 14, wherein the operator device is networked wirelessly to the system controller.

19. A method of controlling decontamination of a substantially enclosed space comprising:
detecting a micro-organism's presence in a substantially enclosed space, wherein the presence of the micro-organism is sensed by one or more sensors that are present within the substantially enclosed space;
alerting a system controller to the presence of the micro-organism in the substantially enclosed space, wherein the system controller is networked to the one or more sensors;
informing an operator device of the presence of the micro-organism in the substantially enclosed space, wherein the operator device is networked to the system controller;
initiating a decontamination process to remove the presence of the micro-organism in the substantially enclosed space, wherein the decontamination process is applied by one or more applicators networked to the system controller, and further wherein the one or more applicators are present in the substantially enclosed space; and
further wherein the system controller initiates the decontamination process by the one or more applicators after the steps of either:
(1) ordering the initiation of a decontamination process from the operate device; or
(2) ordering the initiation of a decontamination process by an event sub-system, wherein the event sub-system is a non-transitory tangible computer-readable medium of claim 14.

20. The method of claim 19, wherein the man-made structure is an office building.
